(19) United States
(12) Patent Application Publication
(10) Pub. No.: US 2007/0082352 A1
(43) Pub. Date: Apr. 12, 2007

(54) MICROSCOPY TIP

(76) Inventor: Peter Jonathan Cumpson, London (GB)

Correspondence Address: Intellectual Property Department;DEWITT ROSS & STEVENS, S.C., Excelsior Financial Centre, 8000 Excelsior Drive, Suite 401, Madison, WI, 53717-1914, US

(21) Appl. No.: 11/531,815

(22) Filed: Sep. 14, 2006

(30) Foreign Application Priority Data
Sep. 15, 2005 (GB) GB 0518867.7

(51) Int. Cl. C12Q 1/68; C12M 3/00

(52) U.S. Cl. 435/6; 435/287.2

(57) ABSTRACT

Disclosed is a tip for use in atomic force microscopy. The tip includes a substrate and a three-dimensional, double-stranded nucleic acid structure attached thereto. The nucleic acid structure may have a single-stranded nucleic acid attached thereto, such as an aptamer sequence. In use, the tip having the nucleic acid structure can be brought into contact with a surface to be imaged.

### CROSS-REFERENCE TO RELATED APPLICATIONS

Priority is hereby claimed under 35 USC §119 to co-pending United Kingdom application Serial No. GB 0518867.7, filed 15 Sep. 2005, which is incorporated herein.

### FIELD OF THE INVENTION

The present invention relates to a tip for use in microscopy, in particular in atomic force microscopy (AFM).

### BACKGROUND

AFM (also referred to as SPM or Scanning Probe Microscopy) is a high-resolution imaging technique that allows researchers to observe and manipulate molecular and atomic level features. A cantilever tip is brought into contact with a surface to be imaged. An ionic repulsive force from the surface applied to the tip bends the cantilever upwards. The amount of bending is measured by a laser spot reflected on to a split photo detector and this is used to calculate the force. If the force is kept constant while scanning the tip across the surface, the vertical movement of the tip follows the surface profile and the surface topography can be recorded by the atomic force microscope. Beyond simple imaging, there is increasing interest in using AFM instruments as analytical tools, i.e. allowing the identification of particular chemical species at a surface via the forces that occur between that species and a “functionalized” AFM tip, i.e. a tip whose surface has a special chemical treatment to make it sensitive to that species. Hence there is now an increasing need for the accurate measurement of small forces by AFM in the mechanical analysis of polymers, unfolding of proteins, biological membranes, electrostatic protein interactions, and ligand-receptor binding studies.

Typically, AFM cantilevers and tips are micromachined, monolithic silicon or silicon nitride. However, silicon tips wear down relatively quickly which results in a loss of sharpness. In any case, even the sharpest tips have an unknown shape on a nanometer scale, which can introduce uncertainties into many types of measurement in which contact area is important.

Recently, there has been much interest in using stiff, functionalized carbon nanotube tips for AFM. Although these tips are stronger, they are difficult to fabricate to optimum length because they are repeated structures. Carbon nanotubes are carbon crystals in this sense. They continue to grow, with the tube getting longer, but section of the tube is the same and growth continues until either the source of atoms is removed or other growth conditions (e.g. high temperature) cease. Accordingly, something must be switched off to ensure a particular length is reached. Often what “seeds” the nanotube takes some time to get started, by which time an adjacent nanotube has already grown too long. It is not possible to control the feedstock supply so as to ensure all the tubes have the required length; some will be short and some long. If a tube is too long, it is too floppy; lateral resolution is lost if it is used on an AFM tip. Although there are ways of “trimming” nanotubes to the required length, it is a skilled and laborious task.

In summary, carbon nanotube tips have several disadvantages. They are non-specific difficult to control, difficult to use, and require complex and involve inflexible chemical preparation methods.

### SUMMARY OF THE INVENTION

The present invention seeks to provide an improved microscopy tip.

According to a first aspect of the present invention, there is provided a tip for use in atomic force microscopy comprising a substrate and a three-dimensional double-stranded nucleic acid structure attached thereto.

An advantage of using such a tip is that double-stranded nucleic acid is sufficiently stiff (persistence length of around 50 nm) and can be designed to self assemble by suitable choice of oligonucleotides. If the persistence length is less than 10 nm, the tip will not work. Double-stranded nucleic acid is a very strong molecule which lasts for decades and carries no chemical safety concerns. When immobilized on an AFM tip, it acts as a very high resolution local chemical or other force probe. A further advantage is that, instead of the irrevocable damage that can be done to a carbon nanotube tip, a nucleic acid structure can be melted and reformed.

Preferably the double-stranded (ds) nucleic acid structure comprises DNA, though the ds nucleic acid may comprise a PNA analog. The ds nucleic acid structure may comprise a polyhedron, and preferably a tetrahedron. The structure may be a polyhedron having up to eight plane surfaces. Higher order polyhedra are probably less stable structurally, but assembly may be easier. The sides of the structure are from about 3 nm to about 10 nm in length, and preferably 5 nm to 10 nm. Each edge is an integral number of double-helical half turns in length.

Alternatively, other structures may be used such as pyramids. The ds nucleic acid structure may be attached to the substrate by thiol interactions. The tip may comprise a gold layer and the ds nucleic acid structure may be attached to the gold layer via thiol interactions.

The substrate is preferably silicon or silicon nitride. The DNA may be attached to the substrate by silane bonding. The substrate may be a cantilever or a cantilever tip.

The probe may comprise a projection from the ds nucleic acid structure which renders the tip chemically sensitive. In other words, it ensures a particular force interaction between the tip and molecules or species at the surface which one wants to detect.

The projection may comprise a single-stranded nucleic acid. The single-stranded nucleic acid may include an aptamer sequence. The single-stranded nucleic acid may be attached to a peptide antibody or bead.

According to a second aspect of the invention, there is provided a method of making a tip for atomic force microscopy including the step of attaching a three-dimensional ds nucleic acid structure to a substrate, whereby the nucleic acid structure is arranged so that in use it can be brought into contact with a surface to be imaged.

Preferably the nucleic acid structure is attached to a cantilever tip. However, the nucleic acid structure may be attached to the cantilever itself.

According to a third aspect of the invention, there is provided a method of atomic force microscopy including bringing a tip comprising a three-dimensional ds nucleic acid structure into contact with a surface to be imaged.

### BRIEF DESCRIPTION OF THE DRAWINGS

FIG. 1 is an example of as “Holliday junction” structure or four way DNA duplex crossover.

FIG. 2 is a representation of a DNA cube showing that it contains six different cyclic strands. Their backbones are shown in red (front), green (right), yellow (back), magenta (left), cyan (top) and dark blue (bottom). Each nucleotide is represented by a single colored dot for the backbone and a single white dot representing the base. Note that the helix axes of the molecule have the connectivity of a cube.

FIG. 3 is a representation of one of the vertices of a DNA tetrahedron. This diagram is topologically correct, but does not attempt to show the 3D geometry of the vertex. Three of the four double DNA strands (A, B and C) form a vertex of the tetrahedron, while the fourth (D) continues for some chosen distance (less than the persistence length) before terminating in a “sticky end.

FIG. 4 is a schematic view of an AFM functionalized self-assembled tip. The rigid double-stranded DNA in a tetrahedron gives structural rigidity; and

FIG. 5 is a top-view of a microfabricated cantilever array with a gold layer designed for attachment of sensor species by thiol groups. The cantilevers are separated by 2 μm from the SiN substrate. These particular cantilevers do not incorporate a microfabricated silicon tip, however, which is the preferred support for the nucleic acid structures we describe.

### DETAILED DESCRIPTION

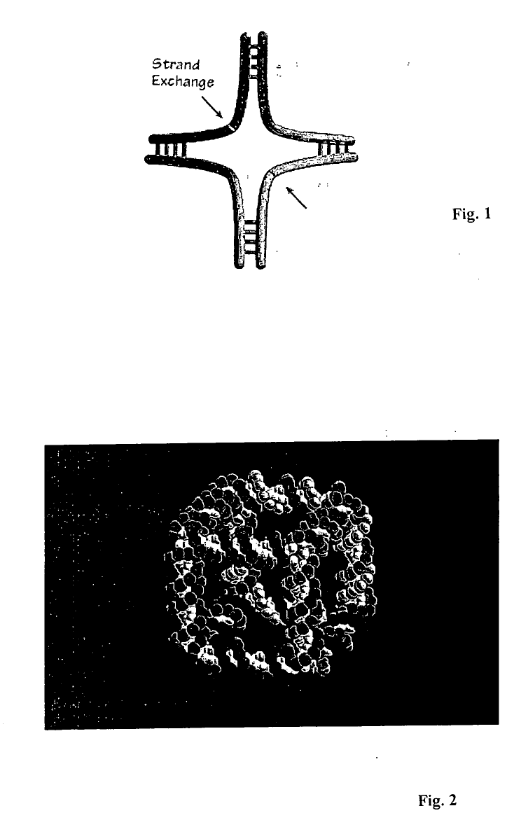

In order to achieve DNA self-assembly, specific oligonucleotide sequences are hybridized under optimum hybridization conditions. DNA combines a high specificity in intermolecular interactions with a large variety of specific binding pairs and is therefore an ideal molecule for the creation of molecular constructs. Two- and three-dimensional structures can be made by self-assembly of synthetic oligonucleotides whose base sequences are designed to control the way in which they hybridise. For example DNA tags have been used to organise the assembly of colloidal particles [1], direct the growth of semiconductor nanocrystals [2, 3] and metal wires [4]. DNA molecules can be used to ensure self-assembly of complex structures, for example by engineering junction structures into otherwise linear molecules (FIG. 1). By this means, DNA polyhedra have been successfully synthesized [5, 6], and simple nanomachines demonstrated [7]. A representation of a DNA cube is shown in FIG. 2. The key to using DNA for this purpose is the design of stable branched molecules, which expand its ability to interact specifically with other nucleic acid molecules. Branched DNA molecules are easy to design, and they can assume a variety of structural motifs. These can be used for purposes both of specific construction, such as polyhedra [10].

The present invention relates to the use of nucleic acid (such as DNA) polyhedra in the self-assembly of nanometer-resolution AFM tips. DNA molecules are here synthesized outside cells and never take part in any biological process; they are used as nanomechanical structures which self-assemble.

The DNA structure is formed before attachment to an AFM tip. Selected single-strand (ss) DNA sequences can be made using commercial DNA synthesis equipment. Sequences can be ordered through a commercial synthesis service offered by a number of suppliers (for example, Integrated DNA Technologies, 1710 Commercial Park, Coralville, Iowa 52241, USA). Single strands of DNA composed of complementary sequences of the bases adenine, cytosine, guanine and thymine (A, C, G and T) hybridise to form a stable duplex (double helix) bound together by hydrogen bonds between complementary base pairs (A±T and C±G).

This product is typically purified before use, such as by high performance liquid chromatography (HPLC). 5′ or 3′ ssDNA ends can be obtained which include:

(a) thiol modifier C6 S—S, for anchoring to a gold coated AFM tip; and/or

(b) biotin, as one method of linking to a molecular recognition group via a biotin-streptavidin linker.

The DNA structure is formed by mixing stoichiometric quantities of strands having specific sequences in buffer (50 mM Na₂HP0₄ at pH 6.5, 1 molar NaCI) at a temperature of 20° C. to give a concentration of around 1 mM. This is performed in solution. The polyhedra (typically tetrahedrons) so formed are anchored on a gold coated AFM cantilever, typically with an existing silicon or silicon nitride tip. Functionalization of some of the ends of these strands with sulphur atoms allows the formation of thiol bonds with the gold coating (e.g. at three corners of a tetrahedron).

Design of the DNA Structure:

(a) Design of Structural Motifs Forming the Polyhedron:

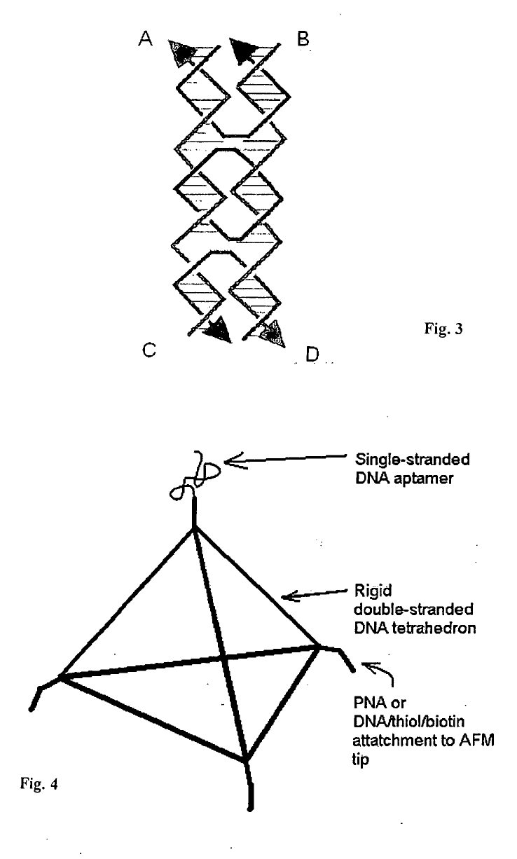

A method of sequence symmetry minimization is used to choose the nucleotide sequences in each of the component strands [11]. Several structurally stable polyhedra may be useful but the simplest is a tetrahedron, each edge of which is a double strand of B-DNA, and each vertex is a junction of a so-called DX molecule. FIG. 3 shows one such junction, i.e. one of the vertices of the tetrahedron. This diagram is topologically correct, but does not attempt to show the 3D geometry of the vertex. Three of the four double DNA strands (A, B and C) form a vertex of the tetrahedron, while the fourth (D) has a specific attachment function; in three of the four vertices of the tetrahedron this is to attach a thiol group to bond with the gold surface of the AFM tip. From the fourth vertex of the tetrahedron this double strand D continues for some chosen distance (less than the persistence length) before terminating in a “sticky end”.

The single-stranded DNA of this “sticky end” can be chosen to have a particular aptamer sequence, or to be functionalized with biotin, peptide or even potentially a molecularly imprinted polymer bead.

An example of a protocol for the preparation of three-dimensional DNA nanostructures is as follows. The tetrahedron-shaped DNA nanostructures are prepared using a set of four complementary oligonucleotides, based on a general approach described by Goodman et al. [Science 310 (2005) 1661-1665], but with particular temperature cycling and concentrations as follows. Gel electrophoresis is used as a method of determining subsequently whether assembly has been successful.

According to a typical protocol, the oligonucleotides are dispersed in TM buffer at a final concentration of 1.0 μm. Starting from a 200 μm stock solution, it is useful to apply a thorough shaking procedure, including vortex and roll shaking when making the dilutions. Equal amounts of the four sorts of oligonucleotides are mixed using the final 1.0 μm solutions. By means of a thermocycler, a temperature treatment is applied as follows. Denaturation is achieved by heating up to 95° C. and holding for 2.5 min. Subsequently, annealing is done by holding the temperature at 55° C. Judging by the results of polyacrylamide-based electrophoresis tests, annealing times of 15 and 30 min deliver very reasonable results: at around 400 bp a single band is observed. On the contrary, the electrophoretograms of samples annealed at temperatures ≦50° C. or ≧60° C., showed additional bands and a higher degree of smear, and these temperatures are therefore deprecated. Also, experiments with too short an annealing period delivered inferior results. In addition to the temperature treatment, the purity of the DNA oligonucleotides was found to be relevant.

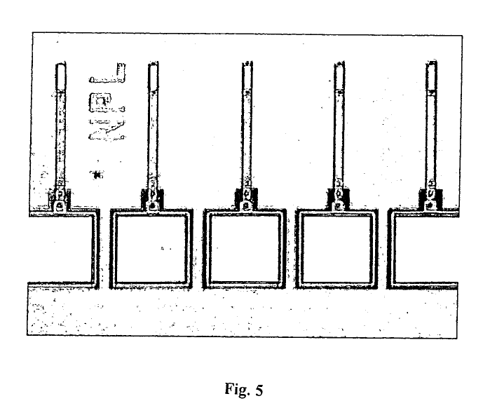

The tip shown in FIG. 4 is a functionalized tip including a DNA aptamer. DNA aptamers are single-stranded nucleic acids that bind particular molecules, proteins or inorganic structures, with high specificity [8,9]. The result is a tightly-bound complex analogous to an antibody-antigen interaction.

In FIG. 4, a single-stranded nucleic acid aptamer is attached to the apex of the ds nucleic acid structure. The simplest addition to the tip is arranged by having one DNA strand from a pair in the ds structure to not end at the tip (apex), but continue with a sequence comprising a spacer sequence and then an aptamer sequence. This is a continuous strand of DNA; the first section hybridises with another strand to form a part of the polyhedron, while the remainder projects from one corner of the polyhedron. The whole length of this strand will typically be between 30 and 120 oligonucleotides in length. By adding a further structure to the ds structure, the tip becomes chemically sensitive. In other words, it ensures a particular force interaction between the tip and molecules or species at the surface which one wants to detect.

In a modification, it is possible to add a further structure to the apex of the ds nucleic acid such as an antibody, a peptide derived from a phage display, or a bead of molecularly-imprinted polymer.

A peptide or bead can be attached in several ways, such as replacing the aptamer sequence with a sequence that hybridizes with a single DNA strand attached to the peptide or bead. Alternatively, a biotin molecule could be attached to the end of the single-stranded DNA that emerges from the polyhedron tip, while attaching streptavidin to the bead or peptide, since biotin and streptavidin bind strongly.

This results in a chemically-sensitive self-assembled device with resolution between about 5 nm and about 50 nm depending on the application. The flexibility and wide applicability of this approach is clear; if one has a method of assembling a tetrahedral structure with a tip that binds to human thrombin protein (for example) replacing the thrombin aptamer with an anthrax aptamer or an MgCl₂ aptamer gives a generic method with very high lateral resolution.

It would be very difficult to achieve this with prior art tips of known structure at the nanometer scale because the aptamer molecule would simply lie down on the surface of the tip and not interact with the molecule or substance one wants to detect.

The present arrangement, as exemplified in FIG. 3, allows chemical force microscopy with generic ability in molecular recognition, and single molecule detection at a surface. Unlike other schemes for chemical force microscopy (CFM), it allows the aptamer (or protein, or molecularly imprinted polymer [MIP] bead) to be presented to the surface under analysis in a controlled orientation, removing many of the problems of uncontrolled orientation typical of “top down” functionalization methods for AFM tips. “Top down” functionalization means that the arrangement of the molecules is by lithography or other processes in which the pattern is somehow “scaled down”, e.g. from a lithography mask or writing by a focused ion beam. The problem with these “top down” methods is that they can control where the molecules are, to some extent, perhaps to within 10 nm or so with the best methods, but they cannot control the orientation of those molecules at the surface. Typically only a small percentage (1 to 10%) will happen to be oriented correctly at the AFM tip to interact with the complementary molecule which one wants to detect at the surface. Often the best one can do is arrange a small “drying stain” at the end of the tip containing molecules that one hopes orient in a useful way. Therefore, only 1 in 10 (or in some particularly difficult cases, 1 in 100) of AFM tips functionalized this way are sensitive. By contrast, the self-assembled DNA structures proposed here have their geometry coded in the DNA sequences of their component strands, which ensures placement and orientation with atomic resolution with respect to this structure (e.g. precisely at the end of the tetrahedron, pointing in a particular direction). The exact location of the entire tetrahedron may not be well controlled, but that does not matter.

(b) Tip Functionalization:

Since this DNA synthesis leads to small quantities of DNA polyhedra in buffer, the preferred method of AFM tip functionalization is as follows. To be suitable for subsequent chemical force studies the AFM cantilevers chosen typically have a spring constant between 0.01 N/m and 1 N/m (for example the “C” microlever from Veeco Metrology Group, a subsidiary of Veeco Instruments Inc., Woodbury, N.Y.). (1) 20-50 nm of gold is evaporated from wire on the underside of the AFM cantilever, i.e. coating the AFM tip, in an Edwards Vacuum system at a base pressure of around 10⁻⁶ mb. We find this typically leaves a series of roughly 10 nm hemispherical excrescences.

(2) The AFM cantilever, on its handling chip, is placed on a UV/ozone cleaned microscope slide, with cantilever uppermost. The tip is therefore separated from the surface of the slide by approximately the thickness of the handling chip. This needs to be done quickly after gold deposition, or some means found to prevent contamination of the gold by atmospheric organic species prior to functionalization. In particular, UV/ozone cleaning of the gold tip itself is not recommended, since surface analysis shows it to give rise to a mixed oxide and carbide layer at the surface.

(3) A sessile drop of solution (around 5 mL, though this is not very critical) containing the self-assembled DNA polyhedra is deposited on the microscope slide and the AFM chip is slowly moved using tweezers so that the tip penetrates the meniscus of the sessile drop.

(4) The slide is washed with fresh buffer, and without allowing the tip to break the meniscus and come back into air, the cantilever is placed in an AFM liquid cell to perform the measurements required. It may also be possible to perform this functionalization in-situ in such a liquid cell.

A large number of polyhedra will attach to the surface but the radius of curvature of the end of the tip is sufficient that only one can fit there. There will be other polyhedra at random locations around the neck of the tip.

FIG. 4 shows a polyhedral dsDNA structure attached to an AFM cantilever. The polyhedron is attached via thiol interactions with a gold coating on the AFM tip. Instead of an AFM tip, the ds DNA structure can be attached to other microfabricated devices, such as the cantilever array in FIG. 5, or other non-cantilever microfabricated structures.

FIG. 5 shows a cantilever array with a gold coating on surfaces. Thiol end groups can be specified, allowing attachment to surfaces. This results in a structurally rigid molecular AFM tip. The calculable nature of the mechanical properties of such a nanotip is extremely useful. The tip has a known shape and allows an accurate determination of the topography of the surface it is used to measure. It can be used to measure inorganic as well as biological structures.

In contrast to carbon nanotube tips, the DNA polyhedron assembles and then no further growth occurs. This is guaranteed by the base sequences of the component DNA strands. Once the polyhedron is complete, there is nothing for a new strand to hybridise with.

Many nanoscale measurements require microfabricated devices with one critical nanoscale feature. We now propose a program for the nanofabrication of these critical features by molecular self-assembly. It is an inexpensive and flexible technique. Examples of potential applications include Nanomechanics using known molecular structures, and therefore quantum-mechanically calculable force constants; Nanowires, using Ag or Pt on self-assembled DNA; DNA scaffolds adsorbed on surfaces as SNOM resolution and fluorescence tests; and Calibration of the length scale in FRET (Fluorescent Resonant Energy Transfer groups can be specified, allowing separations of DNA strands to be measured accurately in the range 0 to about 6 nm).

### REFERENCES

[1] C A Mirkin et al, “A DNA based method for rationally assembling nanoparticles into macroscopic materials”, Nature 382 (1996) 607-609
[2] A P Alivisatos et al, “Organization of nanocrystal groups using DNA”, Nature 382 (1996) 609-611
[3] J L Coffer et al, “Dictation of the shape of mesoscale semiconductor nanoparticle assemblies by plasmid DNA”, Appl. Phys. Lett. 69 (1996) 3851-3853
[4] E Braun et al, “DNA-templated assembly and electrode attachment on a conducting silver wire”, Nature 391 (1998) 775-778
[5] E Winfree et al, “Design and self-assembly of two-dimensional DNA crystals”, Nature 394 (1998) 539-544
[6] A J Turberfield et al, “Coded self-assembly of DNA nanostructures” Bull. Am. Phys. Soc. 44 (1999) 1711.
[7] B Yurke et al, “A DNA-fuelled molecular machine made of DNA”, Nature 406 (2000) 605.
[8] L C Bock et al, “Selection of single-stranded DNA molecules that bind and inhibit human thrombin”, Nature 355 (1992) 564-566
[9] S Klug and M Famulok, “All you wanted to know about SELEX”, Molecular Biology Reports 20 (1994) 97-107.
[10] N C Seeman and P S Lukeman, “Nucleic acid nanostructures: bottom-up control of geometry on the nanoscale”, Rep. Prog. Phys. 68 (2005)237-270.
[11] N C Seeman J. Biomol. Struct. Dyn., 8 (1990) 573-81.

What is claimed is:

1. A tip for use in atomic force microscopy comprising a substrate and a three-dimensional double-stranded nucleic acid structure attached thereto.

2. A tip as claimed in claim 1 wherein the double-stranded nucleic acid structure comprises DNA.

3. A tip as claimed in claim 1 wherein the double-stranded nucleic acid structure comprises a polynucleic acid analog.

4. A tip as claimed in claim 1 wherein the double-stranded nucleic acid structure is tetrahedral in shape.

5. A tip as claimed in claim 4 wherein the sides of the structure are from about 3 nm to about 10 nm in length.

6. A tip as claimed in claim 1 wherein the double-stranded nucleic acid structure is pyramidal in shape.

7. A tip as claimed in claim 1 wherein the double-stranded nucleic acid is attached to the substrate via thiol interactions.

8. A tip as claimed in claim 1 wherein the substrate comprises a material selected from the group consisting of silicon and silicon nitride.

9. A tip as claimed in claim 8 where the double-stranded nucleic acid structure is attached to the substrate by silane bonding.

10. A tip as claimed in claim 1 wherein the substrate is a cantilever or cantilever tip.

11. A tip as claimed in claim 1 wherein a single-stranded nucleic acid is attached to the double-stranded nucleic acid structure.

12. A tip as claimed in claim 11 wherein the single-stranded nucleic acid is attached to a peptide antibody or bead.

13. A method of making a tip for atomic force microscopy comprising:
attaching a three-dimensional double-stranded nucleic acid structure to a substrate, wherein the nucleic acid structure is dimensioned and configured so that it can be brought into contact with a surface to be imaged.

14. A method as claimed in claim 13 wherein the double-stranded nucleic acid structure is tetrahedron-shaped.

15. A method as claimed in claim 14 wherein the double-stranded nucleic acid structure is prepared using complementary nucleotides subjected to a denaturation step and an annealing step, wherein the annealing step is carried out at from about 50° C. to about 60° C.

16. A method as claimed in claim 15 wherein the duration of the annealing step is from about 15 to about 30 minutes.

17. A method as claimed in claim 15 wherein the oligonucleotides are purified using high-performance liquid chromatography (HPLC).

18. A method as claimed in claim 13 wherein the double-stranded nucleic acid structure is attached to a cantilever tip.

19. A method of atomic force microscopy including bringing a tip comprising a three-dimensional double-stranded nucleic acid structure into contact with a surface to be imaged.
